(19) United States
(12) Patent Application Publication
(10) Pub. No.: US 2010/0152855 A1
(43) Pub. Date: Jun. 17, 2010

(54) EXPANDABLE POROUS MESH BAG DEVICE AND METHODS OF USE FOR REDUCTION, FILLING, FIXATION AND SUPPORTING OF BONE

(76) Inventors: Stephen D. Kuslich, Stillwater, MN (US); John E. Kuslich, Phoenix, AZ (US)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(21) Appl. No.: 12/503,663

(22) Filed: Jul. 15, 2009

Related U.S. Application Data
(63) Continuation of application No. 11/906,755, filed on Oct. 3, 2007, now abandoned.
Application No. 11/906,755 is a continuation of application No. 11/282,910, filed on Nov. 18, 2005.
Application No. 11/282,910 is a division of application No. 10/440,036, filed on May 16, 2003, now Pat. No. 7,226,481.
Application No. 10/440,036 is a continuation of application No. 09/909,667, filed on Jul. 20, 2001, now abandoned.
(60) Provisional application No. 60/219,853, filed on Jul. 21, 2000.

(51) Int. Cl. A61F 2/44

(52) U.S. Cl. 623/17.12

(57) ABSTRACT

A method of treating a compression fracture in a bone comprising the steps of forming a transverse cavity within said bone defined by at least one substantially flat surface lying substantially in a transverse plane formed by and communicating with said transverse cavity, the transverse cavity having a substantially uniform transverse extent and a maximum height, the maximum height being less than said transverse extent and applying a force within said transverse cavity generally normal to said surface to displace said surface and restore said bone to its substantially normal anatomic position.

### CROSS-REFERENCE TO RELATED APPLICATIONS

This application is a continuation of U.S. patent application Ser. No. 11/906,755, filed Oct. 3, 2007, which is a continuation of U.S. patent application Ser. No. 11/282,910, filed Nov. 18, 2005, which is a divisional application of U.S. patent application Ser. No. 10/440,036, filed May 16, 2003, now U.S. Pat. No. 7,226,481, which claims priority to U.S. patent application Ser. No. 09/909,667, filed Jul. 20, 2001, which claims priority to U.S. Provisional Application No. 60/219,853 filed Jul. 21, 2000, the entirety of which is incorporated herein by reference.

### BACKGROUND OF THE INVENTION

This invention relates to methods and devices for correcting bone abnormalities and involves the use of a surgical mesh bag which is inserted into a prepared cavity in bone. The bag is inflated using bone replacement material to expand and fill the cavity.

U.S. Pat. Nos. 5,549,679 and 5,571,189 to Kuslich, describe a device and method for stabilizing the spinal segment with an expandable, porous fabric implant for insertion into the interior of a reamed out disc which is packed with material to facilitate bony fusion. In the present invention, a similar bag is used to correct bone abnormalities including, but not limited to, bone tumors and cysts, tibial plateau fractures, avascular necrosis of the femoral head and compression fractures of the spine.

U.S. Pat. Nos. 5,108,404 and 4,969,888 to Scholten et al., describe a system for fixing osteoporotic bone using an inflatable balloon which compacts the bone to form a cavity into which bone cement is injected after the balloon is withdrawn. The invention requires the use of fluoroscopy to monitor the injection and to help guard against cement leakage through fissures in bone. Unfortunately, such leakage is known to occur in spite of these precautions. Since such leakage may cause serious injury, including paralysis, an improved device and method is needed.

U.S. Pat. No. 5,972,015 to Scribner et al., describes a system of deploying a catheter tube into the interior of a vertebra and expanding a specially configured nonporous balloon therewithin to compact cancellous bone to form a cavity. The Scribner patent approach utilizes a non-porous balloon which is inflated within the bone to cause compression. The cavity thus formed, may then be filled with bone cement. Unfortunately, the bag used by Scribner may be ruptured during expansion to compact cancellous bone due to sharp projections found within the cavity to be expanded. Filling the cavity eventually formed could allow leakage of bone cement out of the bone against vessels or nerves which may cause undesirable complications.

The present invention involves an improvement of all of the previous techniques and avoids complications that could occur with the system of U.S. Pat. No. 5,972,015.

All U.S. patents, applications and all other published documents mentioned anywhere in this application are incorporated herein by reference in their entirety.

The art described in this section is not intended to constitute an admission that any patent, publication or other information referred to herein is “prior art” with respect to this invention, unless specifically designated as such. In addition, this section should not be construed to mean that a search has been made or that no other pertinent information as defined in 37 C.F.R. §1.56(a) exists.

### SUMMARY OF THE INVENTION

The invention provides a method of correcting numerous bone abnormalities including bone tumors and cysts, avascular necrosis of the femoral head, tibial plateau fractures and compression fractures of the spine. The abnormality may be corrected by first accessing and boring into the damaged tissue or bone and reaming out the damaged and/or diseased area using any of the presently accepted procedures, or the damaged area may be prepared by expanding a bag within the damaged bone to compact cancellous bone. After removal and/or compaction of the damaged tissue the bone must be stabilized.

In cases in which the bone is to be compacted, the methods and devices of this invention employ a catheter tube attached to an inflatable porous fabric bag as described in U.S. Pat. Nos. 5,549,679 and 5,571,189 to Kuslich, the disclosures of which are incorporated herein by reference. Those bags may be inflated with less fear of puncture and leakage of the inflation medium than thin walled rubber balloons. They may also be used over a Scribner balloon to protect the balloon from breakage and eventually seepage.

The devices of U.S. Pat. Nos. 5,549,679 and 5,571,189 to Kuslich, additionally provide the surgeon with the advantage of safely skipping the first balloon inflation steps of Scribner and Scholten, by expanding the bag through introduction of fill material, such as a bone repair medium thereby correcting the bony defect and deformity and stabilizing it in one step of the procedure.

As indicated above, the damaged bone may be removed by any conventional reamer. Examples of reamers are described in U.S. Pat. No. 5,015,255; U.S. patent application Ser. No. 09/782,176, to Kuslich et al., entitled “Expandable Reamer” and filed Feb. 13, 2001; and U.S. patent application Ser. No. 09/827,202 to Peterson et al., entitled “Circumferential Resecting Reamer Tool,” filed Apr. 5, 2001, the disclosure of which has been expressly recited herein at the end of this application. Other examples of reamers are known and may be used. After the damaged bone or tissue has been removed, bone repair medium may then be inserted into the cavity thus formed, via a catheter and expandable fabric bag as described in U.S. Pat. Nos. 5,549,679 and 5,571,189.

Alternatively, either a smaller than desired cavity may be formed into the bone to be enlarged by compaction or the cavity may be formed only by compaction through introduction of fill material into the bag. In either case, the bag may be positioned over the inflation balloon which is then inflated within the bone site to provide the degree of compaction required. The bag may then be filled with fill material, such as bone repair medium while the balloon remains in place within the bag. Alternatively, the balloon may be removed from the bag prior to filing the bag.

### BRIEF DESCRIPTION OF THE DRAWINGS

A detailed description of the invention is hereafter described with specific reference being made to the drawings in which:

FIG. 1 is a side elevational view of a vertebra that is fractured and in need of repair;

FIG. 2 is a side view of the vertebra of FIG. 1 being reamed out with a reaming tool from the anterior approach;

FIG. 3 is a top view of the vertebra of FIG. 1 showing the reamer forming a pair of cavities within the vertebra from the anterior approach;

FIG. 4 is a side elevational view of the vertebra of FIG. 2 showing placement of an expandable fabric bag of the invention;

FIG. 5 is a top elevational view of the vertebra of FIG. 3 showing a second of two expandable fabric bags of the invention being positioned;

FIG. 6 is a side view of a vertebra being reamed from a posterior approach;

FIG. 7 is a top view of the vertebra of FIG. 6 with a bag in place and a second cavity being reamed;

FIG. 8 is a side elevational view of the vertebra of FIG. 6 with an expandable fabric bag of the invention in place;

FIG. 9 is a top view of the vertebra of FIG. 7 with one bag inflated and the second bag being deployed;

FIG. 10 is a side elevational view showing the vertebra cavity being expanded with an expandable fabric bag about an inflation device in cross-section;

FIG. 11 shows the bag system of FIG. 10 with the vertebra in phantom to show the bag system;

FIG. 12 is a view similar to FIG. 10 showing a different approach to the interior of the vertebra;

FIG. 13 is a view similar to FIG. 11 showing the approach of FIG. 12;

FIG. 14 shows the bag of FIG. 12 in a closed, filled and expanded position;

FIG. 15 is a top view of the bag system of FIG. 12 being inflated through a catheter tube;

FIG. 16 shows a femoral head with avascular necrosis;

FIG. 17 shows the femoral head of FIG. 16 being reamed out;

FIG. 18 shows placement of a bag system of the invention within the cavity in the femoral head;

FIG. 19 is a side elevational view of a tibial plateau fracture;

FIG. 20 is a side view of the fracture of FIG. 19 with a cavity being formed with a reamer; and

FIG. 21 shows the tibial plateau fracture repaired with an expanded inflatable fabric bag in place.

### DETAILED DESCRIPTION OF THE PREFERRED EMBODIMENTS

In the following detailed description, similar reference numerals are used to depict like elements in the various figures.

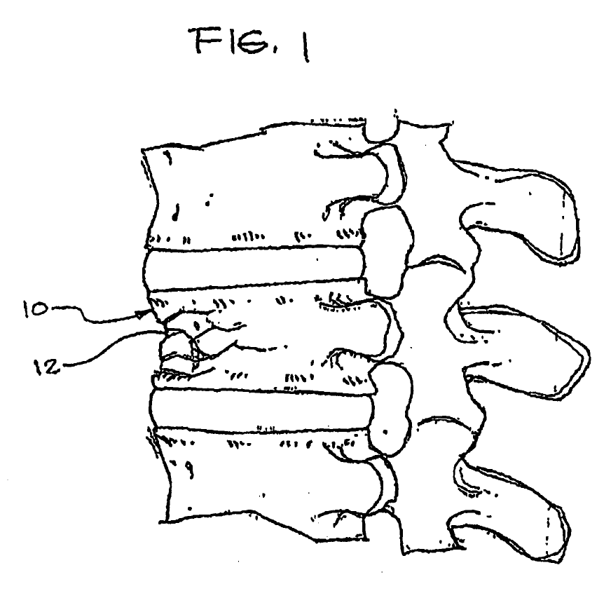

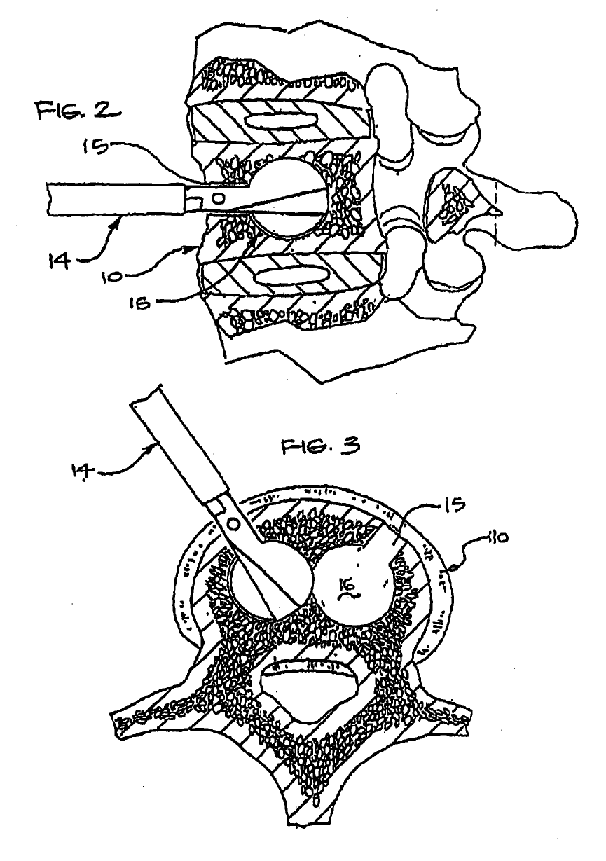

FIG. 1 shows a typical vertebra 10 having compression fractures 12 that is in need of repair. As indicated above the damaged portion of the vertebra 10 may be reamed out, compacted, or otherwise repaired. For example, FIG. 2 shows a reamer 14 entering the vertebra 10 anteriorly to make an opening 15 and cavity 16. Alternatively, multiple cavities 16 may be formed such as is shown in FIG. 3.

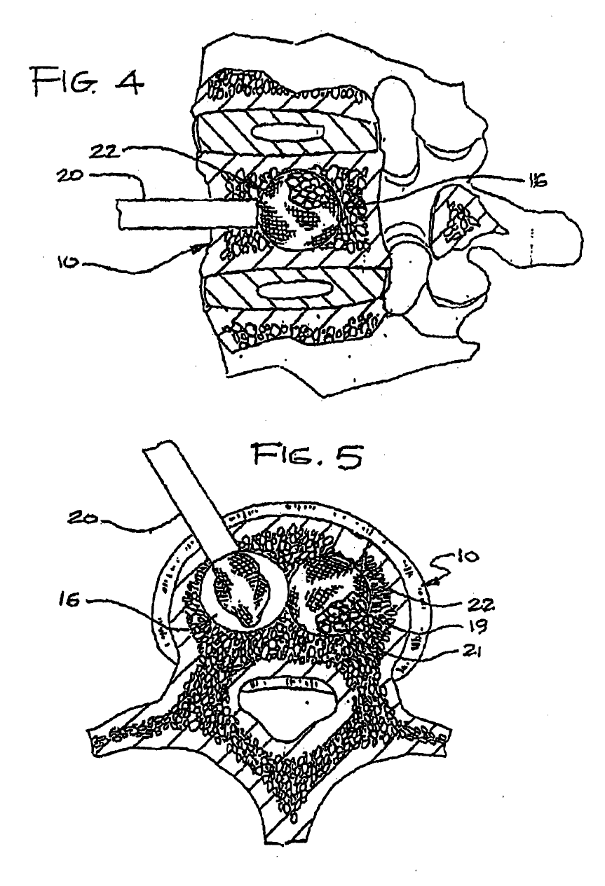

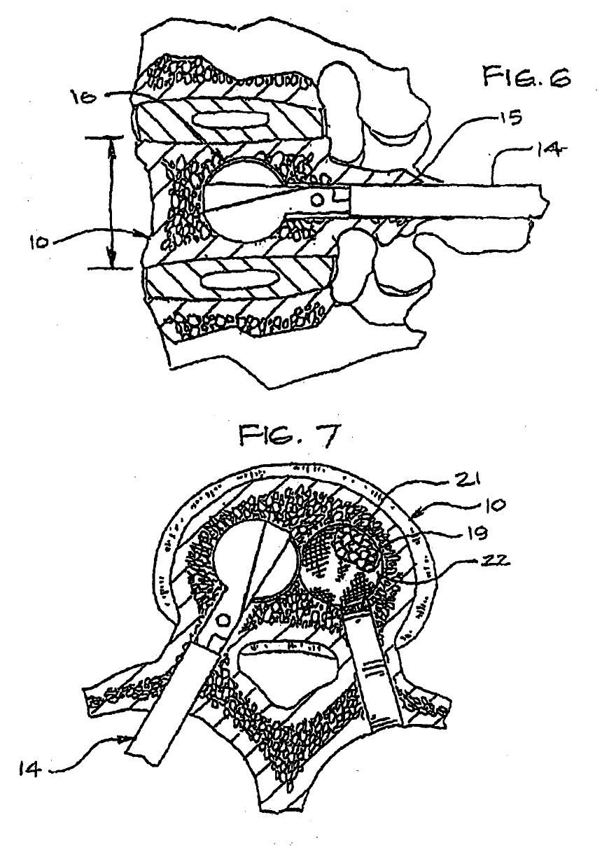

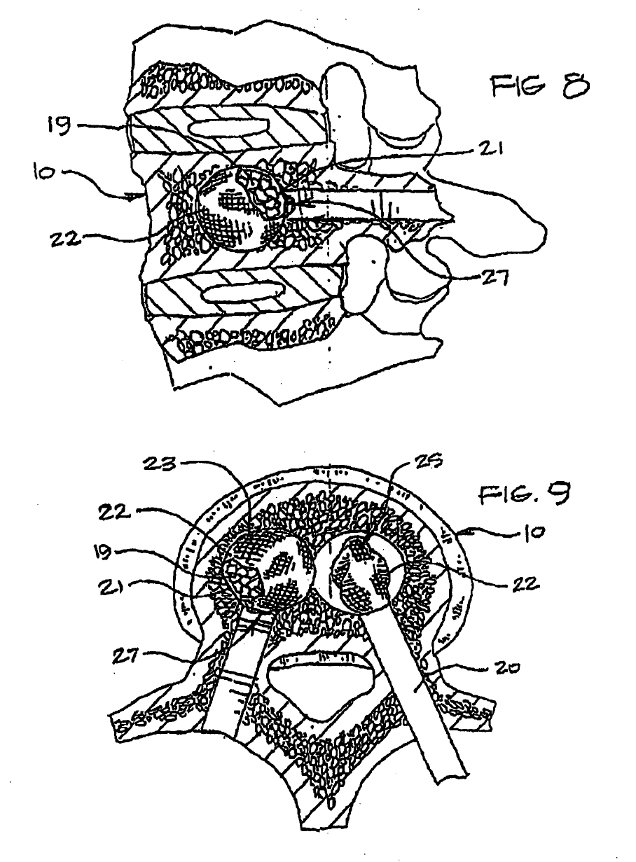

As previously mentioned, the damaged portion of the vertebra 10 may be compacted in addition to or instead of being reamed out. In FIG. 4, a delivery tube or catheter 20 is seen in the process of delivering an expandable fabric bag 22 into the vertebra 10 or into a cavity 16 present therein. As indicated, the cavity 16 may have been created through reaming, compaction by the bag 22 or other device, or by other means. Once the bag 22 is positioned within the vertebra 10, the bag 22 may be inflated or expanded to the limits of the cavity 16 thus formed through insertion or injection of fill material 19 into the interior 21 of the bag 22.

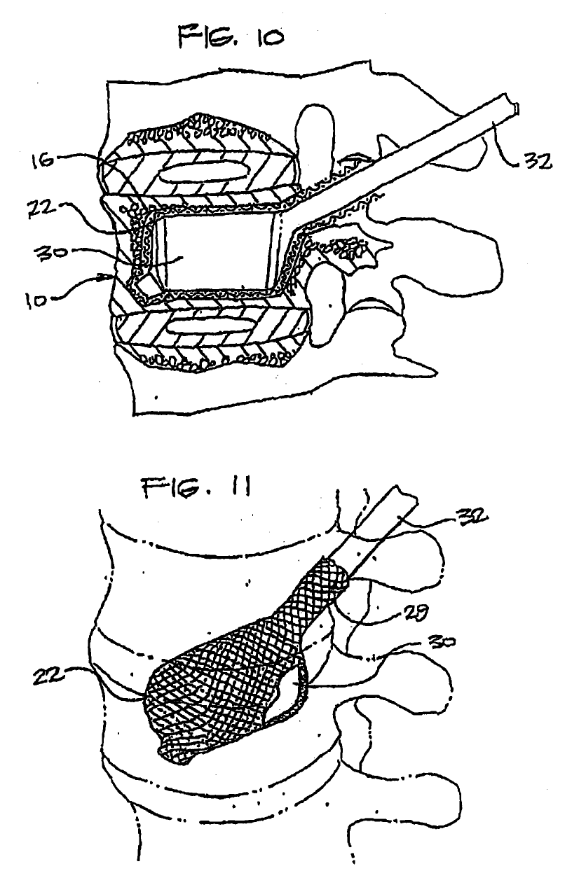

FIG. 5 shows a single filled expandable fabric bag 22 in place with a second expandable bag which is being inserted and expanded within the cavity 16.

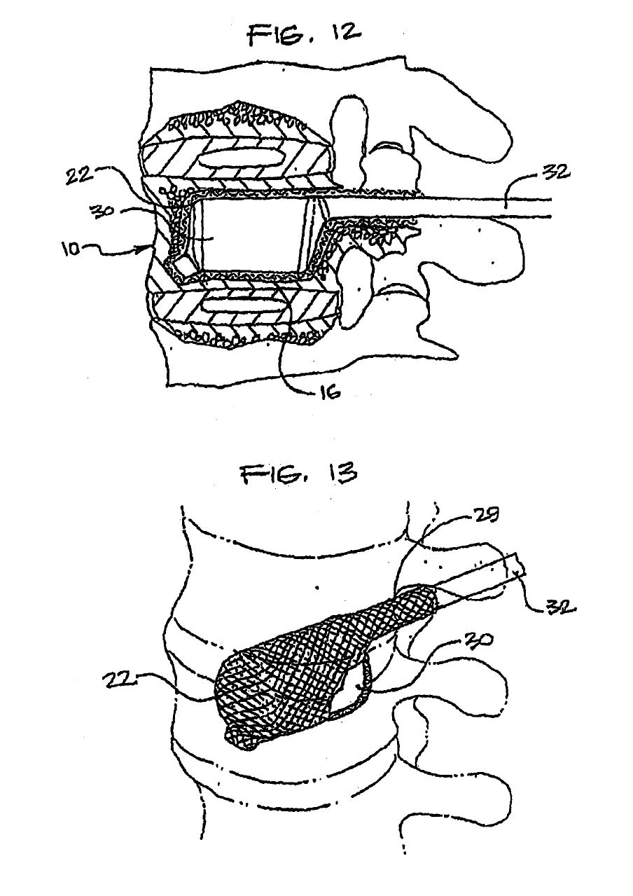

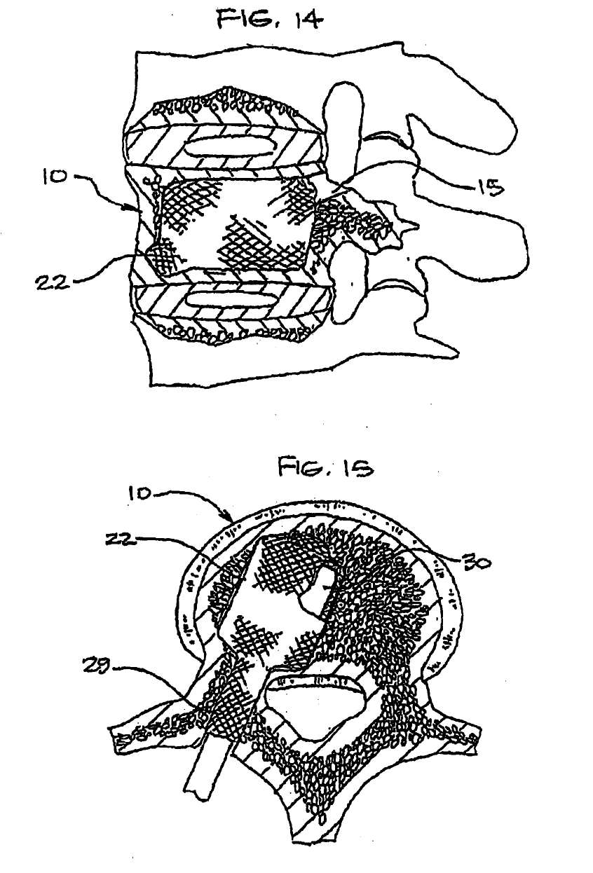

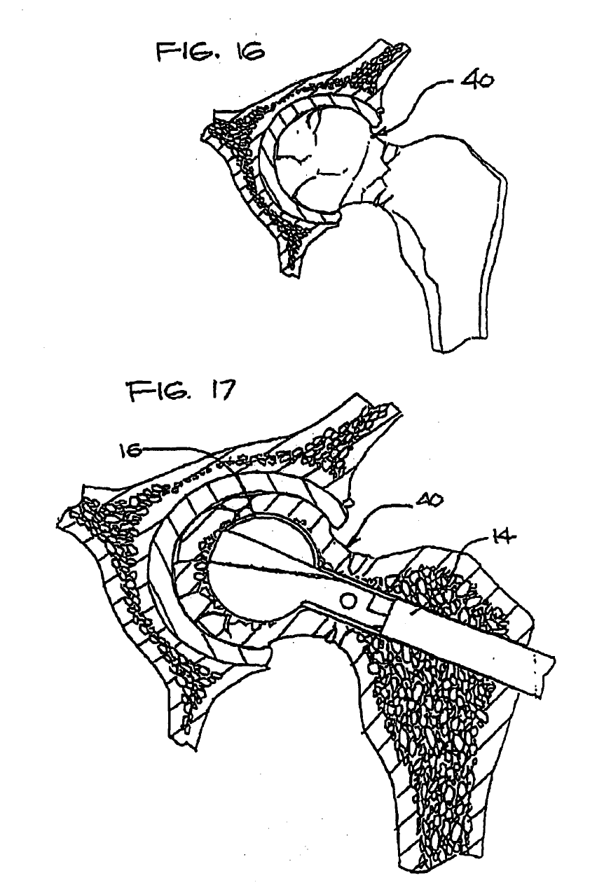

FIGS. 6-9 illustrate a procedure in which the opening 15 and cavity 16 are created posteriorly. Regardless of the direction through which the vertebra 10 is operated on, in all forms, the cavity 16 which is formed is then filled with acceptable bone replacement material.

Bone replacement material 19 may be one or more of the following, or any other biocompatible material judged to have the desired physiologic response:

A) Demineralized bone material, morselized bone graft, cortical, cancellous, or cortico-cancellous, including autograft, allograft, or xenograft;

B) Any bone graft substitute or combination of bone graft substitutes, or combinations of bone graft and bone graft substitutes, or bone inducing substances, including but not limited to: Tricalcium phosphates, Tricalcium sulfates, Tricalcium carbonates, hydroxyapatite, bone morphogenic protein, calcified and/or decalcified bone derivative; and

C) Bone cements, such as ceramic and polymethylmethacrylate bone cements.

The bone replacement material is inserted into the bag 22 via a needle, catheter 20 or other type of fill tool. The bone replacement material expands the bag to the limits of the cavity 16.

The inventive bag 22 may be a small fabric bag, from about one to about four cm in diameter, being roughly spherical in shape, although other elliptical shapes and other geometric shapes may be used. The bag is pliable and malleable before its interior space 21 is filled with the contents to be described. The material of the bag 22 may be configured to take on the shape of the cavity in which the bag is placed. While in this initial condition, the bag may be passed, uninflated, through a relatively small tube or portal, perhaps about three mm to about one cm in diameter.

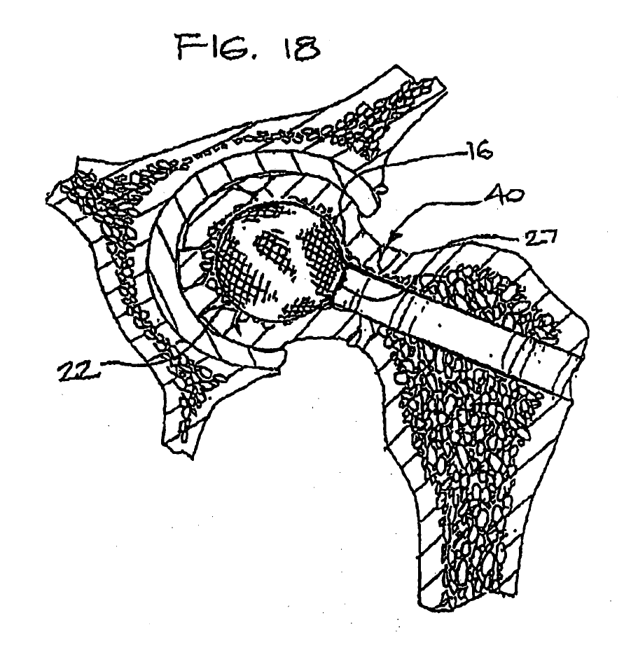

The bag 22, such as may best be seen in FIG. 9, is constructed in a special and novel way. The bag 22 may be constructed of a fabric 23. Fabric 23 may be woven, knitted, braided or form-molded to a density that will allow ingress and egress of fluids and solutions and will allow the ingrowth and through-growth of blood vessels and fibrous tissue and bony trabeculae, but the fabric porosity is tight enough to retain small particles of enclosed material, such as ground up bone graft, or bone graft substitute such as hydroxyapatite or other osteoconductive biocompatible materials known to promote bone formation. The fabric 23 defines a plurality of pores 25. Generally, the pores 25 of the fabric 23 will have a diameter of about 0.25 mm or less to about 5.0 mm. The size is selected to allow tissue ingrowth while containing the material packed into the bag. If bone cement or other material is used which will not experience bone ingrowth, the pores 25 may be much tighter to prevent egress of the media from within the bag 22 out into the cavity 16. This prevents leakage that could impinge upon nerves, blood vessels or the like if allowed to exit the bone.

One or more of the pores 25 may be used as a fill opening 27, wherein the fabric 23 may be manipulated to enlarge a pore to a diameter potentially greater than 5 mm but no more than about 1 cm. Preferably, the fill opening 27 is less than about 5 mm in diameter. Such a pore/fill opening 27 is sufficiently large to allow a catheter, needle, fill tube or other device for inserting or injecting fill material to pass through the fabric 23 and into the interior 21 of the bag 22 without damaging the integrity of the bag 22.

When the bag 22 is fully filled with fill material, the bag will form a self-retaining shape which substantially fills the cavity 16. Once sufficiently full, the fill tool used to place fill material into the bag interior 21 is removed from the opening 27. Where the opening 27 is not a pore 25 but rather a separate and distinct opening in the bag 22, the opening 27 may have a set diameter which requires sealing such as by tying, fastening, welding, gluing or other means of closing the opening 27 after the bag has been filled. Where the opening 27 is a pore 25, upon removal of the catheter or fill tool from the opening 27 the fabric 23 will contract to reduce the diameter of the opening 27 to be substantially similar to that of the other pores 25.

The size and density of the pores determine the ease or difficulty with which materials may pass through the mesh. For instance, very small pores (<0.5 mm) would prohibit passage of all but the smallest particles and liquids. The pore size and density could be controlled in the manufacturing process, such that the final product would be matched to the needs of the surgeon. For example, if methylmethacrylate bone cement were to be used, the pore size would need to be very small, such as about less than 0.5 mm to about 1.0 mm, whereas, when bone graft or biocompatible ceramic granules are used, pore sizes ranging from about 1.0 mm to about 5.0 mm or more may be allowed. The fact that the fabric 23 is properly porous would allow it to restrict potentially dangerous flow of the fill material outside the confines of the bag.

The fabric is light, biocompatible, flexible and easily handled, and has very good tensile strength, and thus is unlikely to rip or tear during insertion and inflation. When the device is inflated, the device expands to fill a previously excavated cavity 16.

The use of the term “fabric” herein is meant to include the usual definition of that term and to include any material that functions like a fabric, that is, the “fabric” of the invention must have a plurality of pores 25 through which material and fluid flow is allowed under the terms as described, and the “fabric” must be flexible enough to allow it to be collapsed and inserted into an opening smaller than the inflated bag size.

The bag 22 need not be woven and may be molded or otherwise formed as is well known in the art. The preferred material may provide the ability to tailor bioabsorbance rates. Any suture-type material used medically may be used to form the bag 22. The bag may be formed of plastic or even metal. In at least one embodiment, bag 22 is formed using a combination of resorbable and/or nonresorbable thread. Bag 22 may include a fill opening 27 which may be a bushing that could be a bioabsorbable and/or nonbioabsorbable plastic, ceramic or metal. The opening 27 may also be hydroxyapatite, or it could be plastic or metal. The opening 27 may also be characterized as a pore 25, wherein a pore 25 of the fabric 23 has been expanded to allow a catheter 20 or other fill device to pass into the interior 21 of the bag 22. The bag 22 could be formed from a solid material to which perforations are added. The bag 22 may be partially or totally absorbable, metal, plastic, woven, solid, film or an extruded balloon.

In embodiments of the present invention a damaged tissue of a body, such as a vertebra 10 may be treated in accordance with the following procedures such as are depicted in FIGS. 1-9.

Initially, the vertebra 10 needing repair is surgically exposed by forming at least one cavity 16. The cavity or cavities 16 may be formed by several different means such as by reaming. Reaming may be accomplished by several means such as including the use of a reamer 14 such as, for example, the Kuslich Expandable Reamer, U.S. Pat. No. 5,015,255, the entire content of which is incorporated herein by reference. Next, the unexpanded mesh bag or Expandable Fabric Bag Device (EFBD) 22 is inserted into the cavity or cavities via catheter 20 or other means. At some point, the fill material 19 is prepared for insertion or injection into the EFBD 22. Following preparation of the fill material 19, the material is injected or otherwise inserted into the bag 22 using sufficient pressure to fill the bag 22 to its expanded state, thus producing rigidity and tension within the cavity or cavities 16 to reach the degree of correction required by virtue of the compression fractures. Finally, the fill opening 27 is closed to prevent egress of inflation material 19.

FIGS. 10-15 show a form of the invention in which a balloon 30 and catheter tube 32 is employed. The balloon 30 is surrounded by an expandable fabric bag 22 to protect the balloon 30 from being punctured during the inflation steps and to remain in place to prevent undesired egress of material injected into the cavity formed in the bone. Balloon 30 may be any medical-grade elastomeric balloon. The balloon 30 may be constructed from latex, urethanes, thermoplastic elastomers or other substances suitable for use as an expandable member. Examples of suitable balloons include, but are not limited to: balloons utilized with the FOGARTY® occlusion catheter manufactured by Baxter Healthcare Corporation of Santa Ana, Calif.; balloons of the type described in U.S. Pat. No. 5,972,015 to Scribner et al., and others. The methods involve placement of the expandable fabric bag 22 of the invention about the balloon 30 of the Scribner et al. device. The expandable bag 22 is left in place before the cavity 16 is filled with bone substitute or bone cement. The expandable fabric bag 22 prevents breakage of the balloon 30 and greatly limits the ability of fill material from leaking out of the cavity through bone fissures where it could cause damage.

As may best be seen in FIGS. 11, 13 and 15, the bag 22 may include a neck 29 which extends outwardly from the bag 22 to completely overlap the shape of balloon 30. The bag 22 and/or balloon 30 may each have a variety of shapes and sizes.

If desired, the expandable fabric bag 22 may be used as the sole inflation device, eliminating the Scribner et al. balloon 30 if the fabric porosity is tight and the inflation media is reasonably viscous.

While many of the previous embodiments have described the use of the bag 22 for repair of tissue such as a spinal body, in FIGS. 16-18 show how the bag 22 may be used in treating avascular necrosis of the femoral head. In FIG. 16, a femoral head 40 is shown which is in need of repair. FIG. 17 shows the femoral head being reamed out with a reamer 14, such as previously described. The reamer 14 forms a cavity 16. In FIG. 18, a bag 22 is shown within the cavity 16 formed within the femoral head 40. The opening 27 of the bag 22 is closed off after being filled and expanded with bone substitute material.

In an alternative embodiment, the Scribner et al. balloon, as previously described, may also be used with the bag 22 for repair of the femoral head 40.

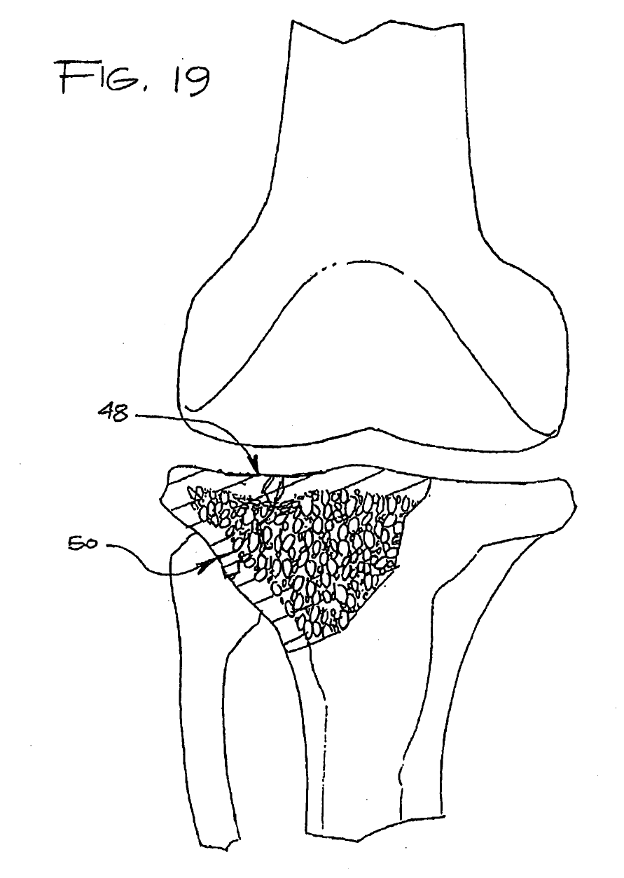

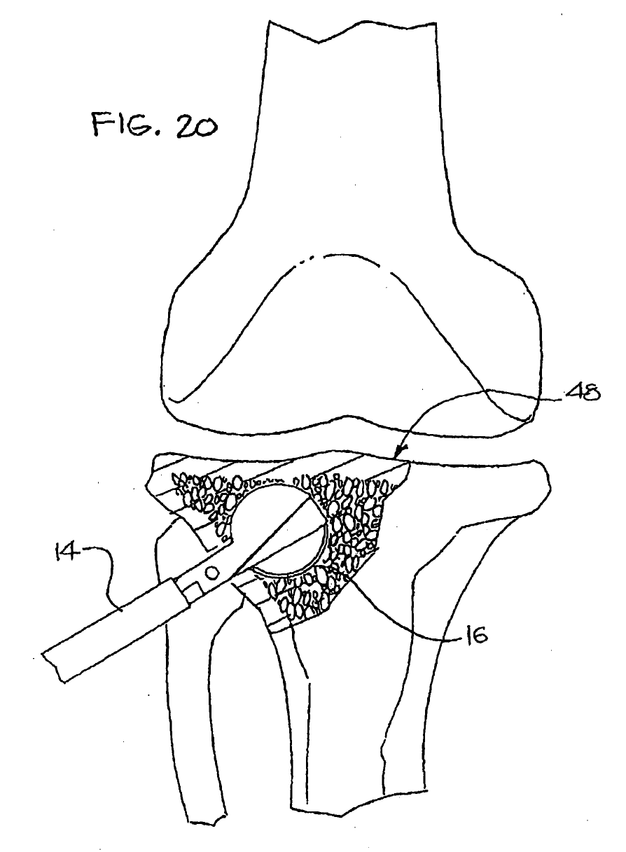

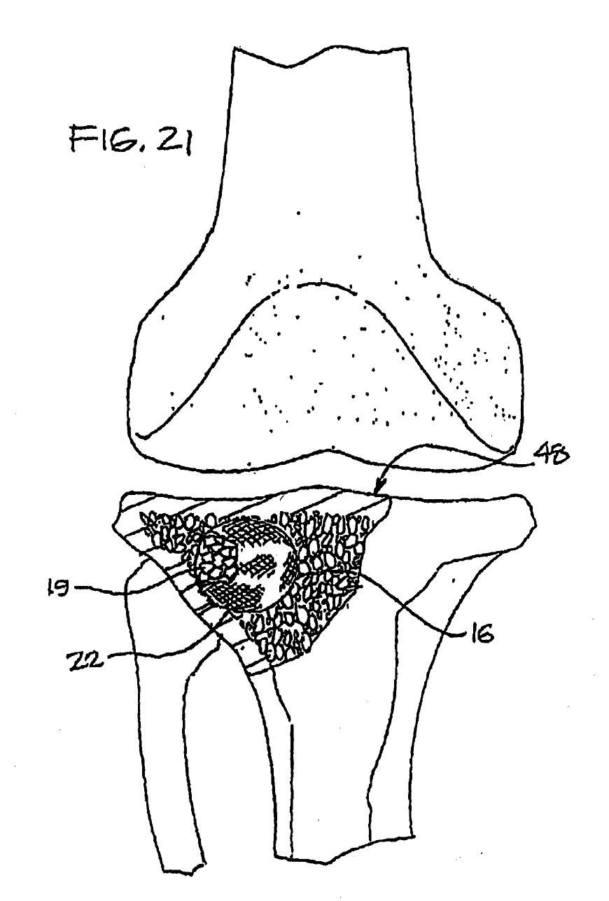

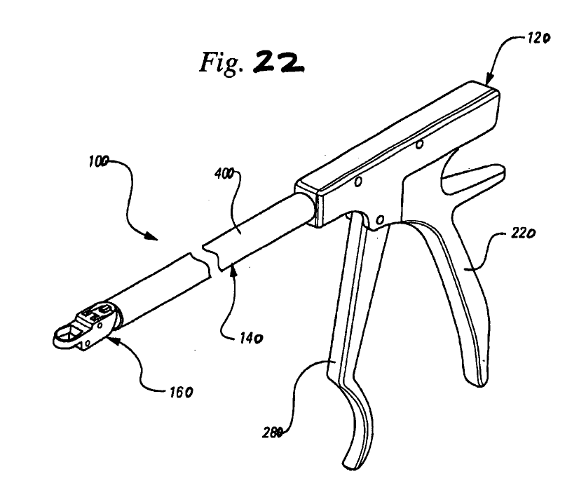

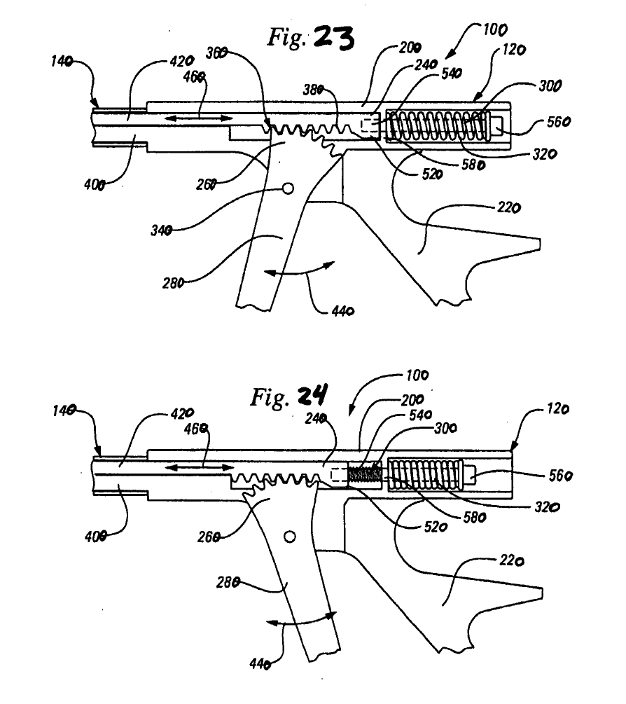

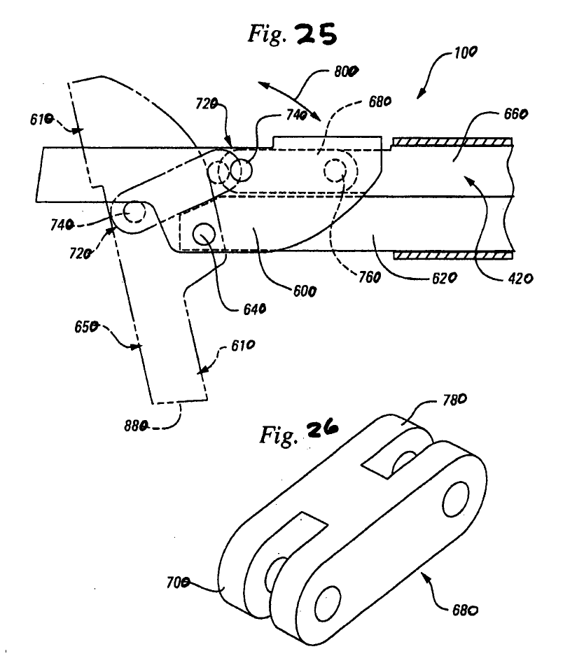

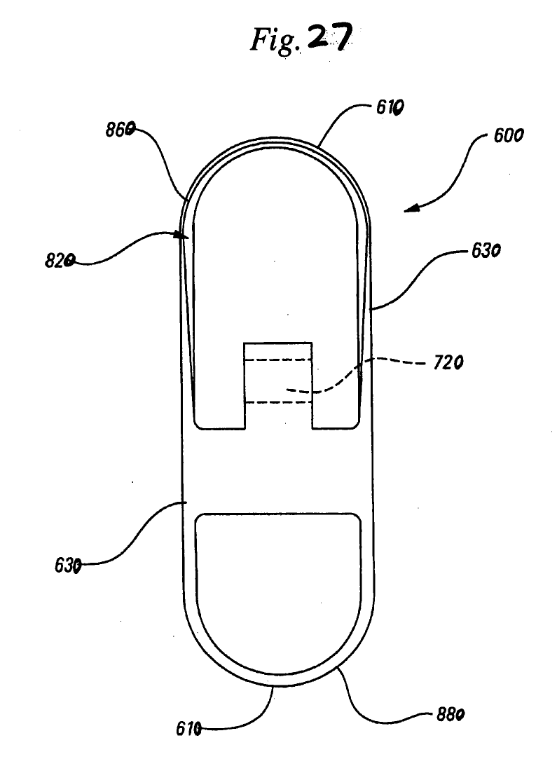

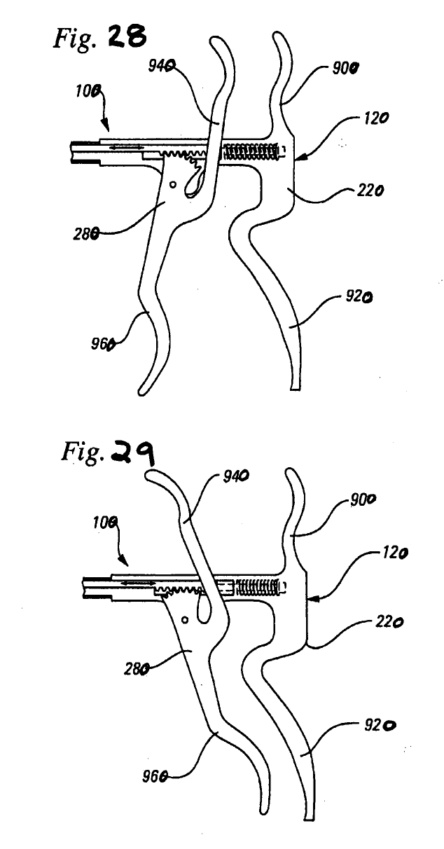

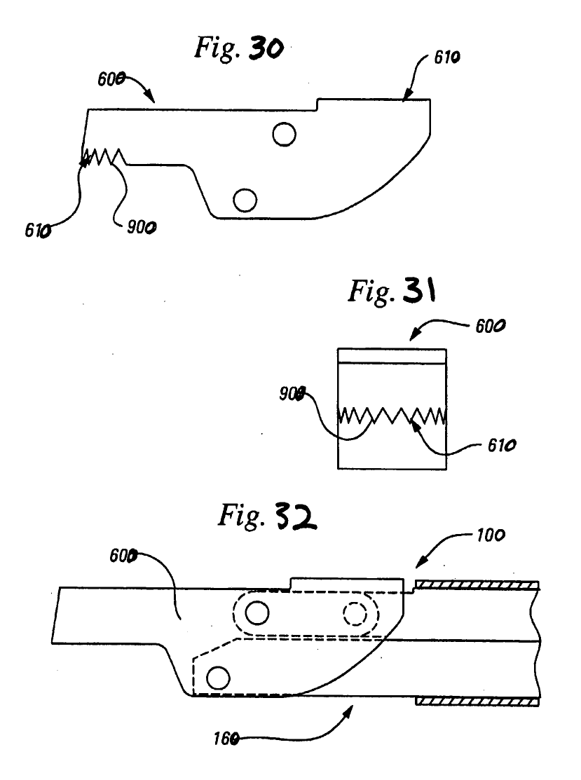

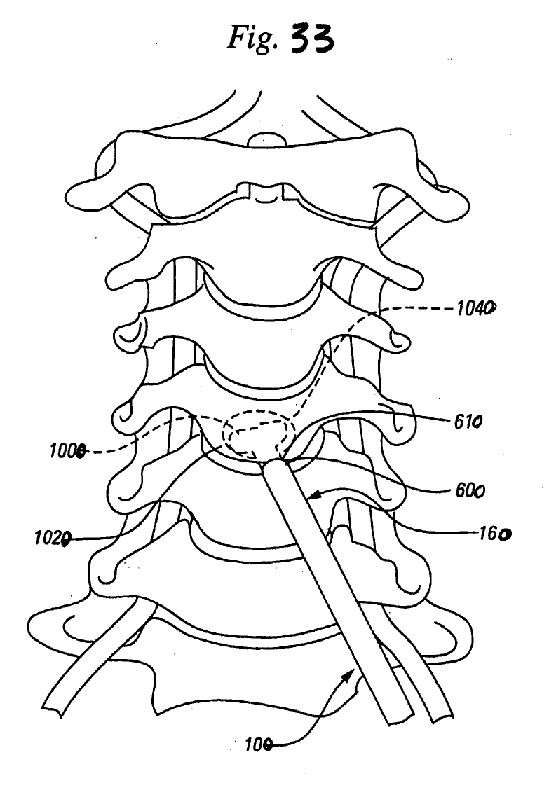

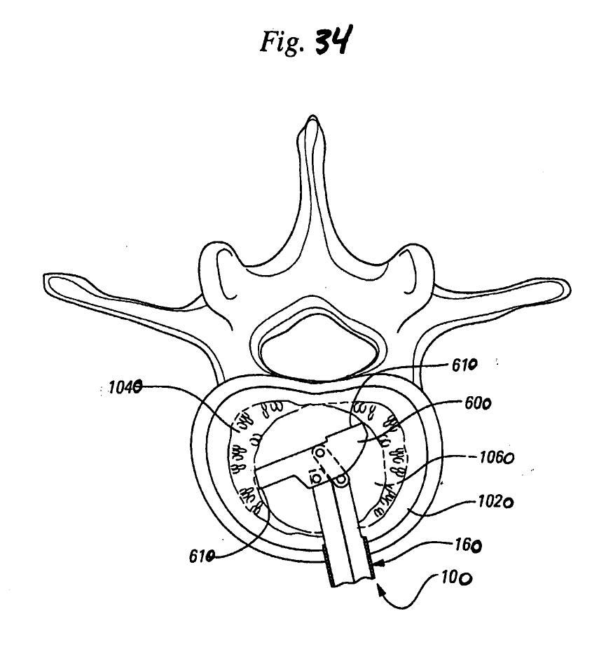

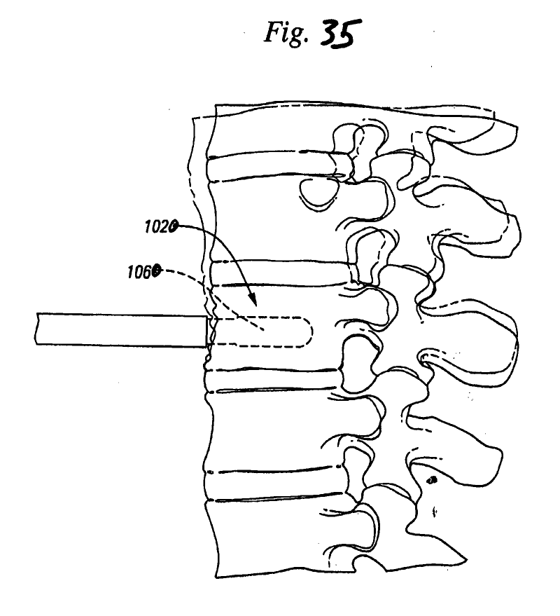

Turning to an embodiment of the invention shown in FIGS. 19-21, a tibial plateau 48 is shown having a fracture 50. The fracture 50 is repaired by forming a cavity 16 with a reamer 14, such as is shown in FIG. 20. As is shown in FIG. 21, once cavity 16 is properly reamed, bag 22 may be inserted therein and filled with bone repair media 19.

Other tissue and bone abnormalities may also be treated with the inventive methods and bag 22 described herein. The present invention is not limited to only treatment of spinal bodies, femoral heads, and tibial plateaus. The bag 22 and the methods of treatment described herein, may be utilized throughout a mammalian body to treat many types of bone and tissue abnormalities including those described herein as well as others.

In addition to being directed to the specific combinations of features claimed below, the invention is also directed to embodiments having other combinations of the dependent features claimed below and other combinations of the features described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below (e.g., claim 3 may be taken as alternatively dependent from claim 2; claim 5 may be taken as alternatively dependent on claim 3, claim 6 may be taken as alternatively dependent from claim 3; claim 7 may be taken as alternatively dependent from claim 3, 5 or 6; etc.).

### BACKGROUND OF THE INVENTION

1. Field of the Invention

This invention relates to an apparatus and method for removing, debriding and/or resecting tissue fragments from a body cavity. In particular, the present invention is directed for use in medical procedures where it may be necessary to remove tissue from a body region. The apparatus and method of the present invention may be especially useful in medical procedures such as orthopedic surgery.

2. Description of the Related Art

Medical procedures involving the removal of tissue from a bone or other region of a body are well known in the art. Of particular interest to the present invention are procedures relating to removal of diseased or damaged tissue of a spinal disk, such as a discectomy.

The spinal disc consists of two types of tissues: the nucleus, and the annulus. The annulus is further divided into the inner and outer annulus. Disc hernias usually consist of a bulge of the nucleus and inner annulus through a rent in a small area of the outer annulus. Partial discectomies are frequently performed when a disc herniation causes pressure on a spinal nerve. The operation consists of removal of the herniated nucleus and portions of the inner annulus. In the past surgeons have used a variety of tools to remove spinal disc tissue during a discectomy. The simplest tools for disc removal are the scalpel and tweezer-type “pick-ups,” which are well known in the art. These tools are very inefficient, as the stringy annular tissues tend to simply move aside and remain attached when these tools are used. Scalpels and pick-ups tend to leave behind fragments of tissue. These fragments can lead to re-herniation—a painful condition that might require a second or even a third operation.

So-called “pituitary rongeurs” and “curettes” are the most frequently utilized instruments. Some examples of these instruments may be seen in the following U.S. Patent references: U.S. Pat. No. Inventor(s): 6,200,320 B1 Michelson 6,142,997 Michelson 5,961,531 Weber et al. 5,766,177 Lucas-Dean et al. 5,653,713 Michelson 5,484,441 Koros et al. 5,451,227 Michaelson 5,312,407 Carter 5,026,375 Linovitz et al. 5,061,269 Muller 4,990,148 Worrick, III et al. 4,777,948 Wright 4,733,663 Farely 4,722,338 Wright et al. 3,902,498 Niederer 3,628,524 Jamshidi 2,984,241 Carlson.

Tools, such as those described in the above cited references, while useful, were not specifically designed to remove disc tissue, and tend to require multiple passes to completely clean out the inner annulus tissue. The use of rongeurs and curettes also tends to leave behind fragments of tissue that may also lead to re-herniation. Furthermore, because these rongeurs and curettes require multiple passes, the operation may be prolonged, possibly leading to increased bleeding and higher infection rates.

Many pituitary rongeurs utilize a single cutting blade at the end of a single, unopposed beam. Actuation of the beam, by means of a drive rod, tends to force the distal shaft to move away from the tissue being cut. An open section in the middle of the beam helps reduce this movement, but does not effectively eliminate the unwanted movement.

Other methods and devices which have been developed in order to improve the effectiveness of a disc removal operation include electrical and laser based cautery. While electrical cautery does effectively destroy disc tissue, it produces heat and smoke in the process. Heat can injure surrounding tissue, including delicate spinal nerves, potentially causing further harm to the patient. In addition, the production of smoke may obscure vision and interfere with the surgeons ability to properly perform the operation. Laser cautery like electrical cautery methods also produce heat and smoke. Low energy lasers tend to be less effective and therefore the disc removal procedure can be prolonged and less than complete. Higher energy lasers produce more heat and smoke and therefore can lead to tissue damage beyond the area of intended removal.

Other devices such as low and high-speed pneumatic or electrical powered rotary burrs are also used. But while they are very useful for removing hard tissues, such as bone, they do not efficiently and effectively remove soft tissues, such as disc material. An example of such a rotary burr is shown in U.S. Pat. No. 5,490,860 to Middle et al., the entire contents of which being incorporated herein by reference. Another type of rotary burr is commercially available and is sold under the name Disc Whisk™ available from Surgical Dynamics Inc. of Norwalk, Conn. Rotary burrs attempt to automate and improve the efficiency of disc removal, but these motorized devices are potentially dangerous when used around the spinal cord and spinal nerves as they develop heat, may grab soft tissue and may penetrate too far.

In light of the above it is clear that there remains a need for an improved, hand-powered tool specifically designed for the removal of diseased soft tissue, such as disc tissue. The current invention improves on the current state of the art by providing a apparatus and method which may be used to efficiently, effectively and safely remove soft tissue from a spinal member such as a disk.

### BRIEF SUMMARY OF THE INVENTION

The present invention is directed to a unique reamer tool that may be used to circumferentially resect tissue from a diseased area of a body. The reamer tool of the present invention consists of a sturdy, yet small diameter, hand powered, multi-bladed cutting tool and its method of use.

In at least one embodiment of the invention the reamer tool has a cutting beam which is pivotally engaged to the tool assembly, a push rod and handle in a rack and pinion relationship to allow the cutter beam to be pivoted relative to the distal end of the tool assembly. The cutter beam may have a plurality of cutting blades or surfaces. As the cutter beam is pivoted as a result of compression of the handle, the cutting blades cut into and resect the surrounding tissue.

In at least one embodiment of the invention the reamer tool may be equipped with a variety of devices designed to make the surgical procedure more efficient. For example the reamer tool may have an attached or integrated suction tube which may be used to remove the tissue which has been resected by the cutting action of the cutter beam. Other devices may also be employed.

### BRIEF DESCRIPTION OF THE SEVERAL VIEWS OF THE DRAWINGS

A detailed description of the invention is hereafter described with specific reference being made to the drawings in which:

FIG. 22 is a perspective view of an embodiment of the invention;

FIG. 23 is a cut-away side view of an embodiment of the invention in the non-actuated position;

FIG. 24 is a cut-away side view of the embodiment of the invention shown in FIG. 2 in the actuated position;

FIG. 25 is a side view of the distal end of an embodiment of the invention wherein the pivoting action of the cutter beam is illustrated;

FIG. 26 is a perspective view of the linkage assembly of the distal end of the reamer tool shown in FIG. 4;

FIG. 27 is a top-down view of an embodiment of the cutter beam;

FIG. 28 is a cut-away side view of a two handed embodiment of the invention in a non-actuated position;

FIG. 29 is cut-away side view of a two handed embodiment of the invention in an actuated, cutting position; F

FIG. 30 is a side view of a serrated cutting beam;

FIG. 31 is an end view of the serrated cutting beam of FIG. 9;

FIG. 32 is an enlarged side view of the end of the tool showing the cutting beam attachment;

FIG. 33 is an anterior view of a spine showing a way in which the present invention may be used, without a guide tube over the tool;

FIG. 34 is a top view of a vertebral body showing one location where the tool can enter and provide reaming; and

FIG. 35 is a side view of a spine section showing an alternative manner in which the present invention may be used.

### DETAILED DESCRIPTION OF THE INVENTION

As may be seen in FIG. 220 the reamer tool, indicated generally at 100 may be thought of as being comprised of three main portions: a proximal portion 120, a middle portion 140, and a distal portion 160.

As may be seen in FIGS. 23 and 24, the proximal or handle portion 120 consists of a handle body 200, a handle body lever 220, a rack 240 and pinion 260, a pinion handle lever 280, a shoulder bolt 300, and a biasing member or return spring 320. The middle portion 140 consists of a shaft tube 400 through which a drive rod 420 is longitudinally actuated. The drive rod 420 is engaged to the distal end 500 (as may be seen in FIG. 25) of the rack 240. When a gripping action supplied by a user (not shown) pivotally actuates the pinion handle lever 280 about the pivot member 340, the teeth 360 of the pinion 260 engage the teeth 380 of the rack 240 resulting in the back and forth movement of the drive rod 420 within the shaft tube 400. As indicated by arrows 440 and 460 the actuation of the pinion handle lever 280 resulting from a compressive force supplied by a user will move the drive rod 420 distally such as shown in FIG. 24, or proximally when the force is removed, as is shown in FIG. 23. The position of the pinion handle lever 280 relative to the handle lever 220, and thus the position of the drive rod 420, will depend on the extent of the compressive force supplied by a user to the pinion handle lever 280 and handle body lever 220.

In FIG. 23 the reamer 100 is shown in the at rest or non-actuated position. The shoulder bolt 300 is engaged to the proximal end 520 of the rack 240. The biasing member or return spring 320 is disposed about a bolt shaft 540 which extends proximally from the rack 240 passing through a return member 580. The bolt shaft 540 ends in an enlarged spring retaining portion 560 of the shoulder bolt 300. The return spring 320 is biasedly engaged between the spring retaining portion 560 of the shoulder bolt 300 and the return member 580. This return spring exerts a force sufficient to keep the drive rod 420 extended distally. The force exerted by the return spring 320 is overcome when the pinion handle lever 280 is engaged by the gripping action of the user previously described and shown in FIG. 24. When the user's grip is relaxed the force exerted by the return spring 320 against the spring retaining portion 560 and the return member 580 will place the reamer back in the at rest position shown in FIG. 23. The tool is returned to the rest position so that its profile is small enough to be removed from a guide tube or a hole in bone.

The lever may be actuated by an air cylinder, an electric solenoid or any other actuator means. Hand operated levers are shown which are less expensive and easier to clean. In the embodiment shown in FIGS. 22-24, the proximal end 120 contains only one handle body lever 220 and one pinion lever 280. This embodiment is designed for single-handed operation. However, in at least one alternative embodiment, shown in FIGS. 28 and 29 the reamer tool may be designed for two-handed actuation. As may be seen, a two handed reamer tool 100 has a the proximal end 120 having a handle body lever 220 which is equipped with opposing grip portions 900 and 920, as well as a pinion lever 280 having opposed section 940 and 960 as well. The present embodiment of the reamer tool 100 may be designed in such a manner that in order to rotate the cutter 600 an two handed grip of alternating action is required to actuate the opposing grips and lever sections 900, 940 and 920, 960 respectively.

Turning to FIG. 25, the distal portion or end 160 of the reamer 100 contains the reamer head or cutting beam 600. The beam has a plurality of cutting surfaces 610. In the embodiment shown, the cutting blades are located at the both ends 860 and 880 of the beam. The beam 600 is pivotally connected to a handle body extension 620 by a lower pivot member 640.

The beam 600 is also engaged to the a distal end 660 of the drive rod 420 via linkage assembly 680. The linkage assembly 680 comprises a pair of beam engagement projections 700, as best shown in FIG. 26, which are disposed about the linkage tab 720 of the beam 600, as best shown in FIG. 27. As may be seen in FIG. 25, a proximal pivot member 740 passes through the linkage tab 720 and the beam engagement projections 700. As may be seen in FIG. 26, the linkage assembly 680 also includes a pair of rod engagement projections 780. As shown in FIG. 25, a distal pivot member 760 passes through the pair of rod engagement projections 780 as well as the distal end 660 of the drive rod 420. As indicated by arrows 800, the unique arrangement of the beam 600 to the drive rod 420 and extension 620 via the linkage assembly 680 provides the reamer 100 with the ability to rotate the beam 600 about the lower pivot member 640 when the drive rod 420 is distally extended in the manner previously described. When the beam 600 is rotated, the cutting edges 610 will cut into and abrade any tissue which is encountered by the moving cutting edges 610.

As may be seen in FIG. 27, the cutting edges 610 are positioned on both ends 860 and 880 of the beam 600 and may be on opposing sides of the beam 600, such as may be seen in FIG. 25. In the present embodiment shown in FIG. 27, the cutting blades 610 may be curved about the shape of a semi-circle, however, the blades 610 may also be provided with other shapes as desired. In addition, the entire perimeter 820 of the beam 600, or a portion thereof, may include bladed portions 610 which extend beyond the semi-circle shape to form a “U” shape, such that cutting may occur along the lateral edges 630 of the perimeter 820 as well as the semi-circular ends 860 and 880. As a result, the reamer 100 may be configured to provide a variety of cutting options which will provide a smooth uniform resecting action as the beam 600 rotates back an forth as indicated by arrows 800 in FIG. 25.

In another embodiment of the invention the beam 600 may include one or more backward cutting blades 650, as is shown in FIG. 25, allowing cutting in both the forward and reverse directions.

The reamer 100 of the present invention may be used in a number of different manners as may be recognized by those of skill in the art. When employed to debride an intervertebral disc, it may be understood that the reamer 100 may be used in the following manner.

After adequate exposure of a small portion of the disc is accomplished by the surgeon using well known standard techniques, any appropriately sized standard drill may be used to perforate the disc. The drill is guided in a direction that crosses the central portion of the disc, to a depth that comes close to, but does not penetrate the far side of the disc.

The distal end 160 of the reamer 100 is then placed into the disc to the full depth of the drilled hole. The reamer 100 is oriented such that its beam 600, with attached cutting blades 610, is parallel to the transverse plane of the disc.

The application of a manual compression force, such as by gripping the pinion lever 280 toward the handle body lever 220 forces the drive rod 420 in the distal direction. This causes the beam 600 to rotate in an elliptical manner around the lower pivot member 640. As is shown in FIG. 25, the beam 600 may be pivotally displaced at least 90 degrees when the pinion lever 280 is actuated such as may be seen in FIG. 24. The cutter will typically provide more than 100 degrees of cutting. This motion causes the cutting blades 610 (and 630) to move against any intervening tissue, cleanly cutting that tissue. The return spring 320 forces the drive rod 420 and the beam 600 back to their original and respective non-actuated positions when the pinion lever 280 is relaxed, such as may be seen in FIG. 23. This procedure may be used to remove the outer nucleus as well as the inner annulus of a spinal disk, leaving the outer annulus intact. Such a procedure is the goal of a partial discectomy. The reamer 100 may then be reoriented 180 degrees, so that the opposite side of the disc can be debrided.

In addition, to providing the cutting motion described above, the present invention may also utilize a variety of blade types to provide for different cutting and resecting characteristics. For example, in FIGS. 4 and 6 the cutter beam 600 may be seen to employ one or more straight edge blades on the cutting edges 61. Alternatively, one or more of the cutting edges 610 may also have serrated teeth 900 such as may be seen in FIGS. 30 and 31.

As may best be seen in FIG. 32, when the reamer tool 100 is in the at rest or non-actuated position, the cutter beam 600 is maintained in a position such that the distal end 160 retains a profile substantially less than the distal end would have when in the actuated position such as is shown illustrated in phantom in FIG. 25. The reduced profile of the non-actuated distal end is sufficiently small to allow insertion of the distal end 160 into a small space or cavity 100 such as is shown in FIG. 33.

In FIGS. 33-34, the reamer tool 100 is seen in use in merely one of a myriad of potential uses. As presently shown, the distal end 160 of the reamer tool 100 may be inserted into an opening or cavity 1000 of a spinal body 1020. As the cutter beam 600 is actuated, such as previously described, the cutting surfaces 610 abrade the surrounding tissue 1040 to form a transverse cavity 1060. Alternatively, the reamer tool 100 may be used to resect tissue from a spinal body 1020 in the middle of a vertebral compression fracture, such as may best be seen in FIG. 35.

After the cavity has been formed, the tool 100 along with any resected tissue is removed. The newly formed cavity may then be filled with filler material such as bone cement and/or graft material. The cavity created by the tool would tend to place the filler in a position where it could accumulate and develop pressure that would tend to elevate or re-expand (or reduce—in orthopedic terms—) the fracture, thereby forcing bone fragments into their pre-injury positions as illustrated in FIG. 35.

In addition to the uses described above, the various embodiments of the reamer tool 100 as described herein may also be used in a wide variety of other procedures. For example, the present reamer tool may be used for removing bone cement from the intramedullary canal of long bones during reconstructive procedures such as joint replacement. The tool may also be useful for debriding cartilage from joints during arthroscopic procedures. Another use may involve using the present reamer tool for certain types of joint arthrodesis, e.g. ankle, inter-tarsal, metatarsal-phalangeal, etc., wherein the tool is used in debriding and preparation of surfaces.

Other uses for the present invention may include: using the reamer tool for producing or sculpting channels for tendon insertion and/or reattachment, such as anterior curciate or rotator cuff repairs. The reamer tool may be used in nasal or sinus surgery for sub-mucosal resections. The reamer tool may also find use in certain gynecological procedures such as a dilation and curettage procedure (D&C). Yet another potential use for the present invention would be for fat immobilization during lipo-suction operations. In such a use the tool could be useful in freeing up fatty tissue to improve removal.

In addition to being directed to the embodiments described above and claimed below, the present invention is further directed to embodiments having different combinations of the features described above and claimed below. As such, the invention is also directed to other embodiments having any other possible combination of the dependent features claimed below.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is:

1. A vertebral augmentation device which comprises:
a) a flowable vertebral augmentation composition for injection within a vertebral body repair site, the flowable composition comprising:
(i) biocompatible, optionally osteoconductive, support elements that are resistant to deformation or fracture under the normal physiologic loads to which the repair site is subject, the support elements being incorporable into the repair site, wherein the support elements are fabricated from ceramic, metal the metal being stainless steel, cobalt-chrome alloy, titanium-nickel alloy, titanium-nickel-aluminum alloy, tantalum or combination thereof, preformed solid polymer, or combination thereof (ii) an amount of biocompatible carrier sufficient to render the augmentation composition flowable, the carrier having the capacity to be cleared from the repair site; and,
b) a containment device, the containment device accepting the flowable vertebral augmentation composition.

2. A vertebral augmentation device which comprises:
a) a flowable vertebral augmentation composition for injection within a vertebral body repair site, the flowable composition comprising: (i) biocompatible, optionally osteoconductive, support elements that are resistant to deformation or fracture under the normal physiologic loads to which the repair site is subject, the support elements being incorporable into the repair site; (ii) an amount of biocompatible carrier sufficient to render the augmentation composition flowable, the carrier having the capacity to be cleared from the repair site wherein the carrier is glycerol; and,
b) a containment device, the containment device accepting the flowable vertebral augmentation composition.

3. A vertebral augmentation device which comprises:
a) a flowable vertebral augmentation composition for injection within a vertebral body repair site, the flowable composition comprising: (i) biocompatible, optionally osteoconductive, support elements that are resistant to deformation or fracture under the normal physiologic loads to which the repair site is subject, the support elements being incorporable into the repair site; (ii) an amount of biocompatible carrier sufficient to render the augmentation composition flowable, the carrier having the capacity to be cleared from the repair site wherein the carrier contains a hydrated polysaccharide; and,
b) a containment device, the containment device accepting the flowable vertebral augmentation composition.

4. A vertebral augmentation device which comprises:
a) a flowable vertebral augmentation composition for injection within a vertebral body repair site, the flowable composition comprising: (i) biocompatible, optionally osteoconductive, support elements that are resistant to deformation or fracture under the normal physiologic loads to which the repair site is subject, the support elements being incorporable into the repair site; (ii) an amount of biocompatible carrier sufficient to render the augmentation composition flowable, the carrier having the capacity to be cleared from the repair site wherein the carrier contains a hydrated unmodified or modified starch or cellulose; and,
b) a containment device, the containment device accepting the flowable vertebral augmentation composition.

5. A method for treating a defect site associated with a vertebral body which comprises introducing within the defect site of a vertebral augmentation device comprising:
a) a vertebral augmentation composition comprising: (i) biocompatible, optionally osteoconductive, support elements that are resistant to deformation or fracture under the normal physiologic loads to which the repair site is subject, the support elements being incorporable into the repair site; and (ii) an amount of biocompatible carrier sufficient to render the augmentation composition flowable, the carrier having the capacity to be cleared from the repair site; and,
b) a containment device, the containment device accepting the vertebral augmentation composition; wherein the vertebral augmentation composition is introduced into the defect site though a needle or cannula and wherein the containment device is fitted on a distal end of the needle or cannula, the containment device occupying the vertebral repair site as the containment device is filled with vertebral augmentation composition.

6. The method of claim 5 wherein the containment device is constructed from a resorbable material.

7. A vertebral augmentation device which comprises:
a) a flowable vertebral augmentation composition for injection within a vertebral body repair site, the flowable composition comprising: (i) biocompatible, optionally osteoconductive, support elements that are resistant to deformation or fracture under the normal physiologic loads to which the repair site is subject, the support elements being osteoinductive demineralized bone and being incorporable into the repair site; (ii) an amount of biocompatible carrier sufficient to render the augmentation composition flowable, the carrier having the capacity to be cleared from the repair site; and,
b) a containment device, the containment device accepting the flowable vertebral augmentation composition.

8. A method for treating a defect site associated with a vertebral body which comprises introducing within the defect site of a vertebral augmentation device comprising:
a) a vertebral augmentation composition comprising: (i) biocompatible, optionally osteoconductive, support elements that are resistant to deformation or fracture under the normal physiologic loads to which the repair site is subject, the support elements being incorporable into the repair site; and (ii) an amount of biocompatible carrier sufficient to render the augmentation composition flowable, the carrier having the capacity to be cleared from the repair site; and, (iii) at least one osteoinductive substance; and
b) a containment device, the containment device accepting the vertebral augmentation composition.

9. The method of claim 8, wherein the osteoinductive substance is demineralized bone.

10. A vertebral augmentation device which comprises:
a) a flowable vertebral augmentation composition for injection within a vertebral body repair site, the flowable composition comprising: (i) biocompatible, optionally osteoconductive, support elements that are resistant to deformation or fracture under the normal physiologic loads to which the repair site is subject, the support elements being incorporable into the repair site; (ii) an amount of biocompatible carrier sufficient to render the augmentation composition flowable, the carrier having the capacity to be cleared from the repair site; and,
b) a containment device, the containment device accepting the flowable vertebral augmentation composition, wherein the containment device is constructed from a resorbable material.

11. A method for treating a defect site associated with a vertebral body which comprises introducing within the defect site of a vertebral augmentation device comprising:
a) a vertebral augmentation composition comprising: (i) biocompatible, optionally osteoconductive, support elements that are resistant to deformation or fracture under the normal physiologic loads to which the repair site is subject, the support elements being incorporable into the repair site; and (ii) an amount of biocompatible carrier sufficient to render the augmentation composition flowable, the carrier having the capacity to be cleared from the repair site; and,
b) a containment device, the containment device accepting the vertebral augmentation composition, wherein the containment device is constructed form a resorbable material.
